(19) United States
(12) Patent Application Publication
(10) Pub. No.: US 2008/0249479 A1
(43) Pub. Date: Oct. 9, 2008

(54) Liquid Drug Delivery System

(75) Inventors: Freddy Zinger, Ra'anana (IL); Igor Denenburg, Riga District (LV)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: MEDIMOP MEDICAL PROJECTS LTD., Ra'anana (IL)

(21) Appl. No.: 11/997,569

(22) PCT Filed: Jul. 31, 2006
(86) PCT No.: PCT/IL2006/000881

§ 371 Date: Jun. 6, 2008

Related U.S. Application Data
(60) Provisional application No. 60/703,899, filed on Aug. 1, 2005.

(51) Int. Cl. A61M 5/31

(52) U.S. Cl. 604/244; 604/256

(57) ABSTRACT

Liquid drug delivery system including an actuating connector with a two-part housing reciprocal between an expanded operative state and a compressed operative state. The actuating connector is coupled on an actuated connector with a Normally Closed (NC) valve having an exteriorly accessible end surface with a Normally Closed (NC) port which is opened on manipulation of the actuating connector from its initial expanded operative state to its compressed operative state to enable an initially occluded flow path. The NC valve's exteriorly accessible end surface is dry subsequent to reverse manipulation of the actuating connector and de-coupling the actuating connector from the actuated connector. A range of actuated connectors are available for handling hazardous liquid drugs supplied in pre-filled syringes or requiring reconstitution, and different modes of administration to a subject. Intended modes of administration include inter alia injection, drip feed, intravenous injection, and the like.

### FIELD OF THE INVENTION

The invention pertains to liquid drug delivery systems in general and liquid drug delivery systems for sterile or aseptic handling.

### BACKGROUND OF THE INVENTION

Drugs for the treatment of cancer, cytostatics, and the like, constitute serious health risks to pharmacy staff and oncology nurses that handle and administer them. Carmel Pharma AB, Sweden have developed a range of drug delivery systems commercially available under the trade mark PhaSeal® for the handling and administration of hazardous liquid drugs. PhaSeal® delivery systems can be viewed online at www.phaseal.com.

PCT International Publication No. WO00/35517 entitled Arrangement, Method and Gas Container for Sterile or Aseptic Handling illustrates and describes a fluid control device for use with a vial containing a liquid drug (see FIGS. 2c to 2j). The fluid control device includes a two-part housing having exteriorly accessible surfaces designed to remain dry during and subsequent a liquid drug transfer procedure. The two-part housing includes component parts each having elastically deformable membranes sealingly juxtaposed one against another and through which a puncturing needle passes therethrough.

PCT International Publication No. WO2005/041846 entitled Safety Drug Handling Device illustrates and describes a fluid transfer assembly similar in construction and operation as PCT International Publication No. WO00/35517's fluid control device.

### SUMMARY OF THE INVENTION

The present invention is directed toward liquid drug delivery systems for safe handling and administering hazardous liquid drugs. The liquid drug delivery systems include an actuating connector and at least one actuated connector designed to ensure that they have no exposed wet surfaces posing a health risk. The actuating connector has a two-part housing reciprocal between an expanded operative state and a compressed operative state. The actuating connector is coupled on an actuated connector with a Normally Closed (NC) valve having an exteriorly accessible end surface with a Normally Closed (NC) port which is opened on manipulation of the actuating connector from its initial expanded operative state to its compressed operative state to enable an initially occluded flow path. The NC valve's exteriorly accessible end surface is dry subsequent to reverse manipulation of the actuating connector and de-coupling the actuating connector from the actuated connector. A range of actuated connectors are available for handling hazardous liquid drugs supplied in pre-filled syringes or requiring reconstitution, and different modes of administration to a subject. Intended modes of administration include inter alia injection, drip feed, intravenous injection, and the like.

### BRIEF DESCRIPTION OF THE DRAWINGS

In order to understand the invention and to see how it can be carried out in practice, preferred embodiments will now be described, by way of non-limiting examples only, with reference to the accompanying drawings in which similar parts are likewise numbered, and in which:

FIG. 1 is a pictorial view of a liquid drug delivery system including an actuating connector, an actuated vial connector with a Normally Closed (NC) valve, and an actuated delivery connector with a Normally Closed (NC) valve for use with a syringe, a vial containing a liquid drug, and a delivery tube;

FIG. 2 is a perspective cross section of the actuating connector in its initial expanded operative state along line A-A in FIG. 1;

FIG. 3 is a longitudinal cross section of the actuating connector in its initial expanded operative state along line A-A in FIG. 1;

FIG. 4 is a longitudinal cross section of the actuating connector in its compressed operative state along line A-A in FIG. 1;

FIG. 5 is a perspective view of the actuated vial connector with a transverse cross section of the NC valve along line B-B in FIG. 1;

FIG. 6 is a longitudinal cross section of the actuated vial connector with its NC valve in its closed state along line C-C in FIG. 1;

FIG. 7 is a longitudinal cross section of the actuated vial connector with its NC valve in its open state along line C-C in FIG. 1;

FIGS. 8-10 show an operation cycle of the actuating connector and a NC valve; and

FIGS. 11A-11O show the use of FIG. 1's liquid drug delivery system for administering a hazardous liquid drug to a patient.

### DETAILED DESCRIPTION OF PREFERRED EMBODIMENTS OF THE PRESENT INVENTION

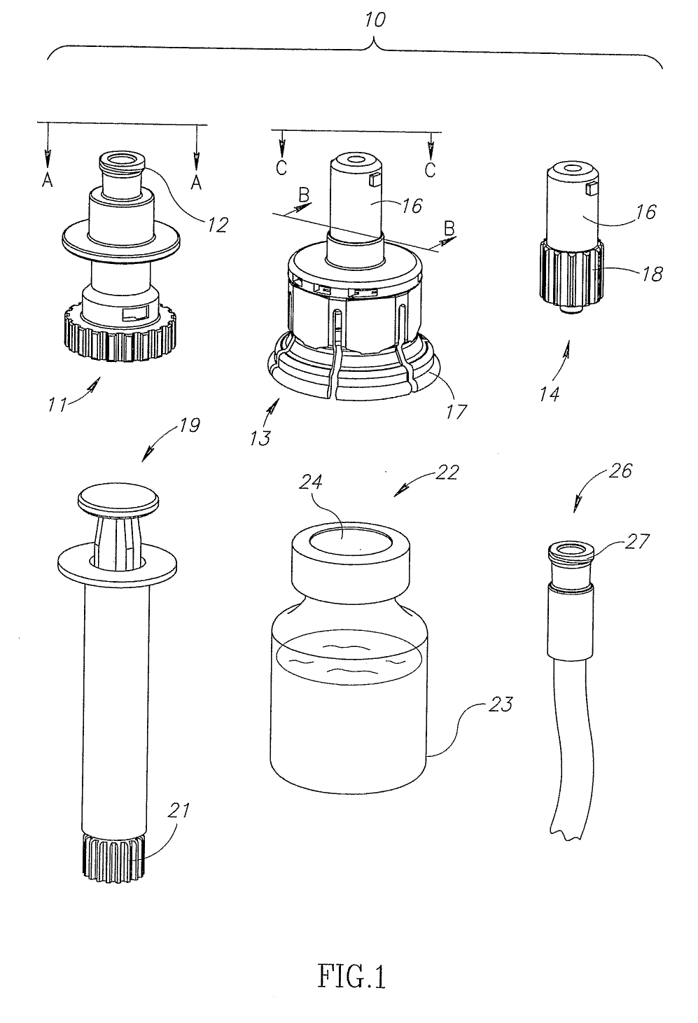

FIG. 1 shows a liquid drug delivery system 10 including an actuating connector 11 with a female Luer fitting 12, an actuated vial connector 13 and an actuated delivery connector 14. The actuated vial connector 13 and the actuated delivery connector 14 both have a Normally Closed (NC) valve 16 for secure removable coupling to the actuating connector 11. Secure removable coupling can be achieved by way of bayonet fittings, and the like. The actuated vial connector 13 includes a NC valve 16 integrally formed with a vented vial adapter 17. The actuated delivery connector 14 includes a NC valve 16 integrally formed with a male Luer lock fitting 18. The liquid drug delivery system 10 is intended for use with a syringe 19 with a male Luer lock fitting 21 for sealing screw thread coupling on the female Luer fitting 12, a hermetically sealed vial 22 containing a hazardous liquid drug 23 and stopped by a rubber stopper 24, and a delivery tube 26 with a female Luer fitting 27 for receiving the male Luer lock fitting 18 for administering the hazardous liquid drug 23 to a patient.

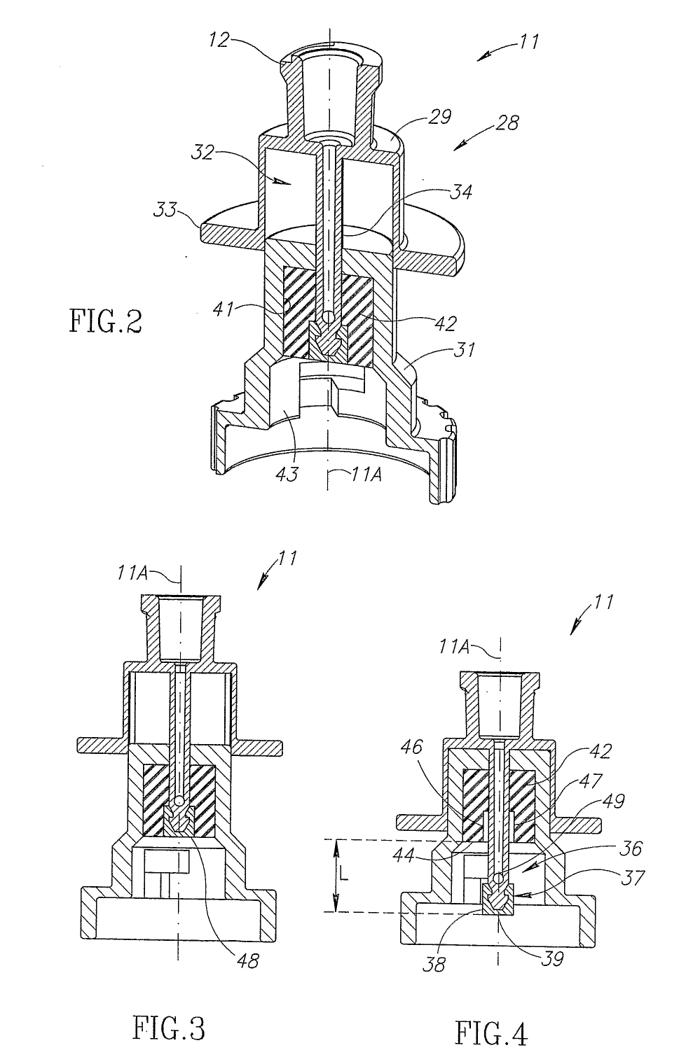

FIGS. 2-4 show the actuating connector 11 having a longitudinal axis 11A and including a two-part housing 28 having a female member 29 for telescopically slidingly receiving a male member 31 on manipulation of the actuating connector 11 from an expanded operative state (see FIG. 3) to a compressed operative state (see FIG. 4). The female member 29 includes the female Luer fitting 12 and a cylindrical chamber 32 with an outward radial flange 33 for assisting a user to reciprocate the actuating connector 11 between its expanded operative state and its compressed operative state. The female Luer fitting 12 is in flow communication with an axially directed tubular plunger 34 having a distal end 36 fitted with a plunger tip 37 (see FIG. 4). The plunger tip 37 has a right cylindrical shape including an axially directed peripheral wiping surface 38 and a distal end surface 39 transverse to the longitudinal axis 11A.

The male member 31 includes a plug chamber 41 for snugly housing a tubular plug 42, and a valve chamber 43 for removably sealingly receiving a NC valve 16. The plug 42 has a generally annular distal end surface 44 transverse to the longitudinal axis 11A and an axially directed recess 46 for sealingly receiving the plunger tip 37 in the actuating connector' expanded operative state. The recess 46 has a right cylindrical wiped surface 47. The plug's distal end surface 44 and the plunger tip's distal end surface 39 are flush in the actuating connector's expanded operative state to form a planar end surface 48 (see FIG. 3). The plunger 34 has a radial port 49 disposed above the plunger tip 37 and which extends beyond the distal end surface 44 in the actuating connector's compressed operative state by a length L (see FIG. 4).

The actuating connector 11 is preferably made out of suitable rigid plastic material except for the plunger tip 37 and the plug 42 which are made out of suitable natural rubber or synthetic rubber-like material for sealing purposes.

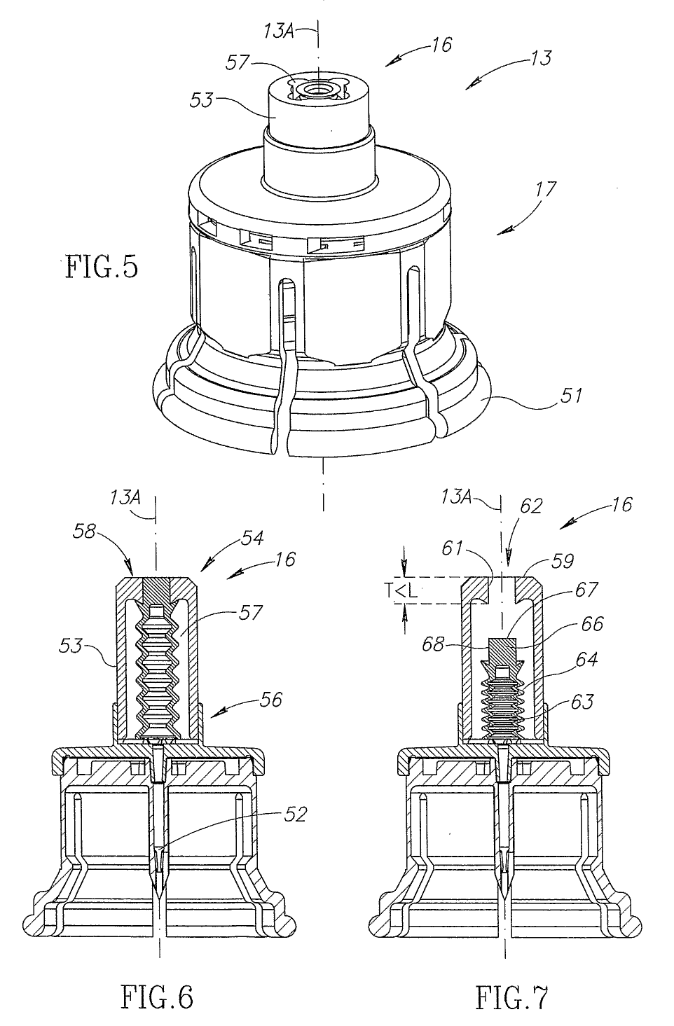

FIGS. 5-7 show the actuated vial connector 13 having a longitudinal axis 13A and including the NC valve 16 in its closed state (see FIG. 6) and its open state (see FIG. 7). The actuated vial connector 13 includes the vial adapter 17 with a resiliently deformable skirt 51 for snapping onto a vial 22 for securely retaining same therein, and a hollow puncturing cannula 52 for puncturing a vial's rubber stopper 24. The NC valve 16 includes an axially directed tubular member 53 with a proximal end 54 sealingly received in the valve chamber 43 on coupling the actuating connector 11 on the actuated connector 13, and a distal end 56. The tubular member 53 has an axially directed bore 57 in flow communication with the cannula 52. The proximal end 54 has an exteriorly accessible end surface 58 transverse to the longitudinal axis 13A. The end surface 58 has an annular surround 59 with an axially directed right cylindrical wiped surface 61, and a Normally Closed (NC) port 62 in flow communication with the axially directed bore 57.

The bore 57 houses a compression spring 63 fitted in a sheath 64 having a stopper 66 for normally stopping the NC port 62. The stopper 66 has a right cylindrical shape, and a proximal end surface 67 transverse to the longitudinal axis 13A and an axially directed peripheral wiping surface 68. The stopper's proximal end surface 67 has the same shape and dimension as the plunger tip's distal end surface 39 for sealing abutment thereagainst in the coupled state of the actuating connector 11 and the actuated vial connector 13. The compression spring 63 urges the stopper 66 into the NC port 62 such that the end surface 58 is planar (see FIG. 6). The annular surround 59 has an axial thickness T where T<L such that coupling the actuating connector 11 on the actuated vial connector 13 and manipulating the actuating connector 11 to its compressed operative state plunges the plunger 34 into the bore 57 to displace the port 49 thereinto for flow communication purposes. The actuated vial connector 13 is preferably made out of suitable rigid plastic material except for the sheath 64 and the stopper 66 which are made out of suitable natural rubber or synthetic rubber-like material for sealing purposes.

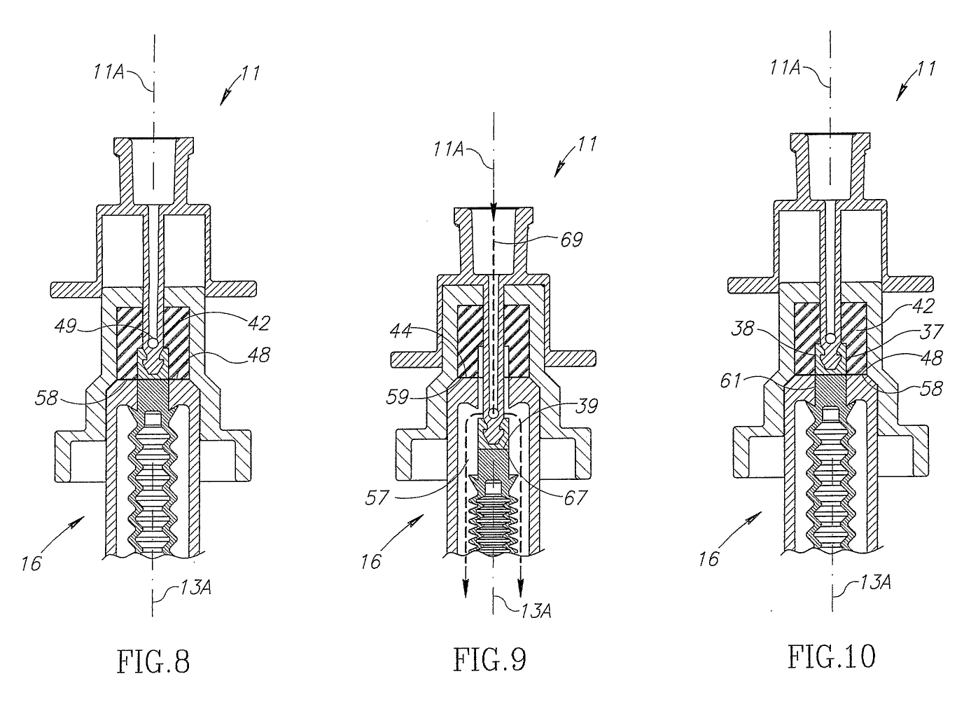

FIGS. 8-10 show an operation cycle of the actuating connector 11 and either the actuated vial connector's or the actuated delivery connector's NC valve 16 for enabling a flow path 69 shown in dashed lines in FIG. 9 and occluding same in FIGS. 8 and 10.

FIG. 8 shows the end surface 48 in concentric sealed abutment with the end surface 58 and the port 49 sealed in the plug 42. FIG. 9 shows manipulation of the actuating connector 11 from its expanded operative state to its compressed operative state for plunging the plunger tip 37 into the bore 57 to establish the flow path 69 between the female Luer fitting 12 and the bore 57. In this position, liquid contents flow along the flow path 69 whilst the plunger tip's distal end surface 39 and the stopper's proximal end surface 67 remain dry by virtue of their sealing abutment against one another. Similarly, the plug's distal end surface 44 and the proximal end's annular surround 59 remain dry by virtue of their sealing abutment against one another.

FIG. 10 shows reverse manipulation of the actuating connector 11 to its expanded operative state for withdrawing the plunger tip 37 back into the plug 42. This withdrawal movement causes the plunger tip's wiping surface 38 to wipe dry the NC valve's wiped surface 61 and then the recess's wiped surface 47 such that any residual liquid upstream of the initial contact between the plunger tip's wiping surface 38 and the NC valve's wiped surface 61 is wiped back into the plunger 34 via the port 49. Thus, the entire planar end surfaces 48 and 58 are both dry on decoupling the actuating connector 11 from the NC valve 16.

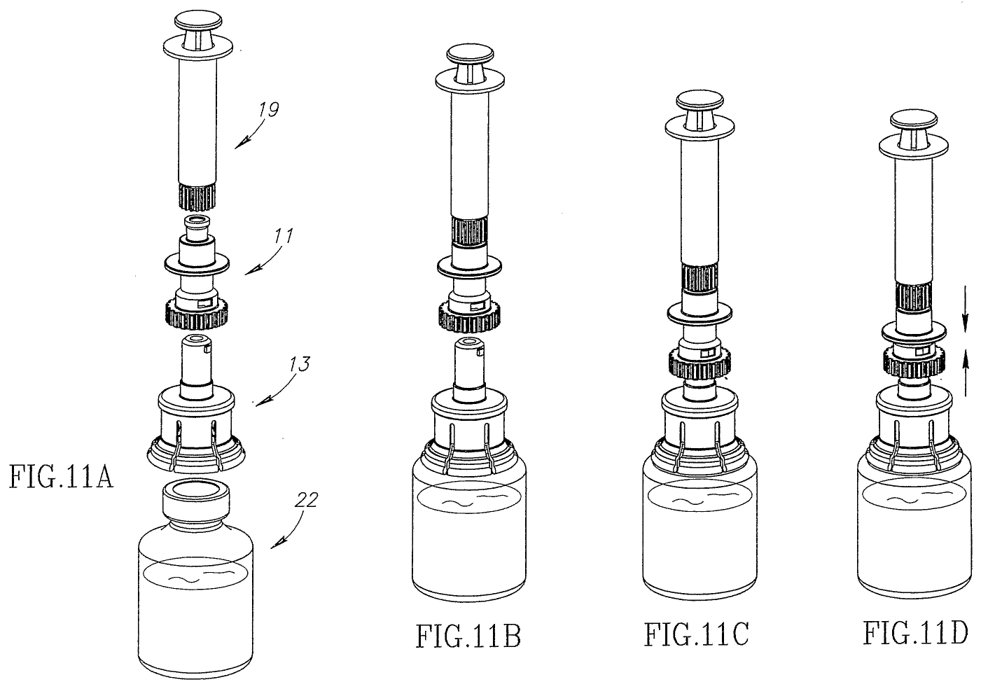

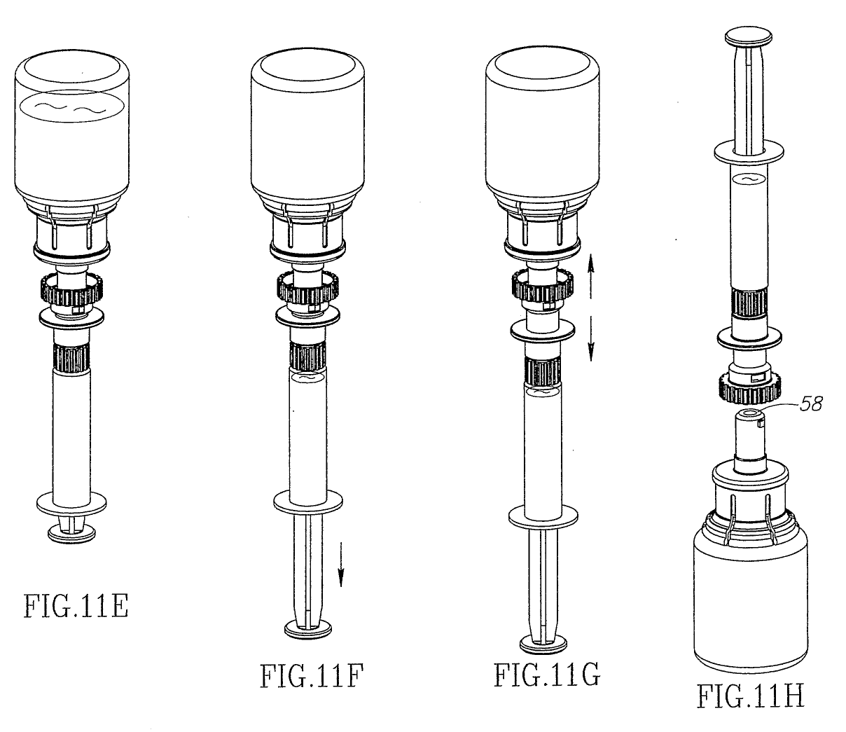

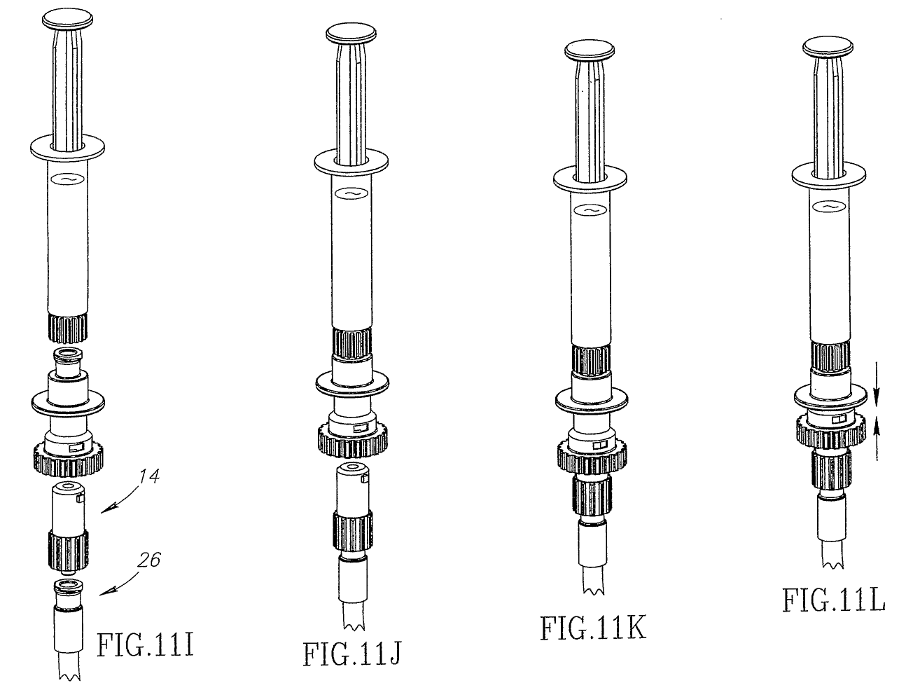

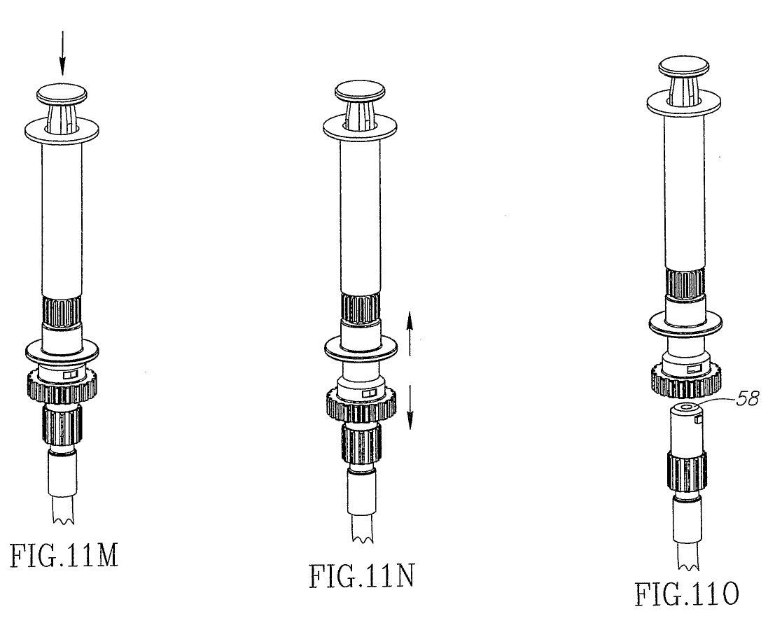

The use of the liquid drug delivery system 10, the syringe 19, the vial 22 and the delivery tube 26 for administering the hazardous liquid drug 23 to a patient is now described with reference to FIGS. 11A-11O.

FIG. 11A shows the actuating connector 11 in its initial expanded operative state, the actuated vial connector 13, the syringe 19, and the vial 22. The user attaches the actuating connector 11 to the syringe 19 and the actuated vial connector 13 to the vial 22 (see FIG. 11B). The user securely couples the actuating connector 11 on the actuated vial connector 13 (see FIG. 11C). The user manually compresses the actuating connector 11 to its compressed operative state which plunges the plunger tip 37 into the actuated vial connector 13 thereby enabling an initially occluded flow path between the syringe 19 and the vial 22 (see FIG. 11D). The user inverts the assemblage (see FIG. 11E) and aspirates the syringe 19 to draw the hazardous liquid drug thereinto (see FIG. 11F). The user manually expands the actuating connector 11 to its expanded operative state which in turn reverts the actuated vial connector 13 to its normally sealed state (see FIG. 11G). The user decouples the actuating connector 11 from the actuated vial connector 13 (see FIG. 11H). The actuated vial connector's entire end surface 58 is dry to the touch. The user can safely dispose the spent vial 22 and the actuated vial connector 13.

The user prepares the actuated delivery connector 14 and the delivery tube 26 for administering the contents of the syringe 19 to a patient (see FIG. 11I). The user attaches the actuated delivery connector 14 to the delivery tube 26 (see FIG. 11J). The user securely couples the actuating connector 11 on the actuated delivery connector 14 (see FIG. 11K). The user manually compresses the actuating connector 11 to its compressed operative state for opening the NC port 62 to enable the flow path 69 between the syringe 19 and the delivery tube 26 (see FIG. 11L). The user injects the syringe 19 to administer the drug (see FIG. 11M). The user manually expands the actuating connector 11 to its expanded operative state to revert the actuated delivery connector 14 to its normal sealed state (see FIG. 11N). The user decouples the actuating connector 11 from the actuated delivery connector 14 (see FIG. 11O). The actuated delivery connector's end surface 67 is also dry in a similar manner as the actuated vial connector's end surface 58. The user safely disposes the spent syringe 19 and the actuating connector 11.

While the invention has been described with respect to a limited number of embodiments, it will be appreciated that many variations, modifications, and other applications of the invention can be made within the scope of the appended claims. For example, plunger tips can have different transverse cross sections than their preferred circular cross section as long as stoppers have matching transverse cross sections. Moreover, a plunger tip and its counterpart stopper may have a convex distal end surface and a matching concave proximal end surface or a concave distal end surface and a matching convex proximal end surface as long as a plunger tip sealingly abuts against a stopper on coupling an actuating connector on an actuated connector.

1. Liquid drug delivery system comprising:
(a) an actuating connector having a two-part housing for reciprocation between an initial expanded operative state for occluding a flow path and a compressed operative state for enabling said flow path,
said two-part housing including a tubular female member having a longitudinal axis, an exteriorly accessible port, a chamber for telescopically slidingly receiving a tubular male member in said compressed operative state, and an axially directed tubular plunger in flow communication with said exteriorly accessible port, said plunger having a distal end with a plunger tip having an axially directed peripheral wiping surface and a distal end surface, and a radial port proximal to said plunger tip,
said tubular male member having a longitudinal axis, a plug chamber for snugly housing a tubular plug and a valve chamber, said tubular plug having a generally annular distal end surface and an axially directed recess for snugly receiving said plunger tip for occluding said radial port in said expanded operative state, said radial port protruding beyond said tubular plug's distal end surface in said compressed operative state; and
(b) an actuated connector having a longitudinal axis and a Normally Closed (NC) valve including an axially directed tubular member having a proximal end sealingly received in said valve chamber on coupling said actuating connector on said actuated connector and a distal end, said axially directed tubular member having an axially directed bore and said proximal end having an exteriorly accessible end surface with a normally closed (NC) port in flow communication with said axially directed bore,
said axially directed bore including a biased stopper for normally stopping said NC port, said stopper having a proximal end surface and an axially directed peripheral wiping surface, said stopper's proximal end surface having the same shape and dimension as said plunger tip's distal end surface for sealing abutment thereagainst in said coupled state,
the arrangement being such that in said coupled state, manipulation of said actuating connector from said initial expanded operative state to said compressed operative state plunges said plunger tip through said NC port to protrude into said rigid tubular member such that said radial port is in flow communication with said axially directed bore for establishing said flow path, and
subsequent reverse manipulation of said actuating connector to said expanded operative state withdraws said plunger tip into said recess to occlude said flow path and effect a wiping action such that said NC valve's exteriorly accessible end surface is dry on de-coupling said actuating connector from said actuated connector.

2. The system according to claim 1 wherein said plunger tip has a right cylindrical shape.

3. The system according to claim 1, wherein said tubular plug's generally annular distal end surface and said plunger tip's distal end surface form a planar end surface in said expanded operative state.

4. The system according to claim 1, wherein said female member includes an outward radial flange for assisting a user to manipulate said actuating connector between said expanded operative state and said compressed operative state.

5. The system according to claim 1, wherein said actuated connector is constituted by an actuated vial connector including a vial adapter integrally formed with said NC valve wherein said vial adapter includes a resiliently deformable skirt for snapping fitting onto a vial and a hollow puncturing cannula in flow communication with said axially directed bore for flow communication with said vial.

6. The system according to claim 1, wherein said actuated connector is constituted by an actuated delivery connector including a male fitting integrally formed with said NC valve and in flow communication with said axially directed bore for administrating a liquid drug.
